(19) United States
(12) Patent Application Publication
(10) Pub. No.: US 2008/0009819 A1
(43) Pub. Date: Jan. 10, 2008

(54) Knitted Loop Tape And Diaper Provided With The Same

(76) Inventors: Jackson Liu, Taoyuan Hsien (TW); Thomas Kuo, Taoyuan Hsien (TW); Shih-Lai Lu, Taipei (TW)

Correspondence Address: 3M INNOVATIVE PROPERTIES COMPANY, PO BOX 33427, ST. PAUL, MN, 55133-3427, US

(21) Appl. No.: 11/570,843

(22) PCT Filed: Jun. 23, 2005
(86) PCT No.: PCT/US05/22605

§ 371 Date: Dec. 18, 2006

(30) Foreign Application Priority Data
Jul. 2, 2004 (TW) 093120020

(51) Int. Cl. A61F 13/15; A44B 11/25; D04B 21/00

(52) U.S. Cl. 604/385.23; 24/306; 604/389; 66/195

(57) ABSTRACT

A knitted loop tape comprises a pressure sensitive adhesive layer; a film backing layer located above the pressure sensitive adhesive layer and bonded thereto; and a loop fabric layer located above the film backing layer and bonded thereto, the loop fabric layer having a first side and a second side opposite to the first side, and comprising a plurality of parallel warp yarns, each warp yarn being formed with a plurality of loops thereon; wherein each loop is respectively oriented toward the first side to define a first angle with respect to the film backing layer and the first angle ranges from about 10° to about 80°.

### BACKGROUND OF THE INVENTION

1. Field of the Invention

The present invention relates to a knitted loop tape adapted to be cut into pieces to form the loop portions for fasteners of the type including releasably engageable hook and loop portions, and to garments such as disposable diapers including pieces of such knitted loop tape.

2. Description of the Relevant Art

In the disposable personal care garment market, more and more customers like hook-and-loop fastening systems comprising a non-woven backsheet, a knitted loop tape and hooks used as a mechanical closure system for products such as disposable diapers, adult incontinence briefs, training pants, sanitary napkins and the like.

Such a fastening system is cloth-like, which is quite different from the traditional tape closure system comprising a polyolefin backsheet, a film frontal tape (FFT) and film side tapes.

However, although consumers like this mechanical closure system, it is high priced mainly resulting from knitted loop tapes. As shown in FIG. 1, a conventional knitted loop tape comprises a pressure sensitive adhesive (PSA) layer A, a pattern printed layer B, a film backing layer C, a loop fabric layer D and a low adhesion backsize (LAB) layer E, wherein one layer bonds to the other from the bottom to the top in sequence.

According to FIGS. 1 and 2, the loop fabric layer is formed with a plurality of loops thereon and each loop lies on the film backing layer in the same direction ((+)Y direction shown in FIG. 2), such that the angle between each loop and the film backing layer (X-Y plane shown in FIG. 2) is substantially zero. When the knitted loop tape is wound into a roll (usually along either the (+)Y direction or the (−)Y direction), if no LAB layer is provided, the PSA layer will stick to the loop fabric layer. Accordingly, when the roll of the knitted loop tape is unwound, a very high unwinding force would occur, such that a de-lamination is possible between the film backing layer and the loop fabric layer also the loops on the loop fabric layer will be damaged.

However, the provision of the LAB layer increases the costs in the manufacturing as well as in the materials, which thus increases the cost of the loop tape.

Further, as previously indicated, the loops of the knitted loop tape are all oriented in the same direction ((+)Y direction shown in FIG. 2). When the tape is disposed along the upper edge of a front portion of the diaper and engages with the hooks provided on the hook portion on each lateral side of the upper edge of a rear portion of the diaper, there is a strong peeling force between loops of the tape and the hooks on one lateral side. However, there is a weak peeling force between loops of the tape and the hooks on the other lateral side of the upper edge of a rear portion of the diaper, since the hook portion on the other lateral side is peeled in an opposite direction. This weak peeling force makes the hook and loop engagement on the other side of the diaper loosen easily.

Therefore, there is a need for a knitted loop tape, which is not expensive and easy to manufacture. Further, there is a need for a diaper provided with a hook-and-loop fastening system, wherein the peeling force on each side of the diaper is similar to each other.

### SUMMARY OF THE INVENTION

It is a primary objective of the present invention to provide a knitted loop tape, wherein no low adhesion backsize layer is required such that the cost in manufacturing and materials can be reduced.

It is another objective of the present invention to provide a knitted loop tape, wherein the de-lamination between the film backing layer and the loop fabric layer can be prevented.

It is yet another objective of the present invention to provide a diaper provided with a hook-and-loop fastening system, wherein the peeling force on each side of the diaper is similar to each other.

To achieve the above objectives, the first preferred embodiment of the present invention, a knitted loop tape, comprises: a pressure sensitive adhesive layer; a film backing layer located above the pressure sensitive adhesive layer and bonded thereto; and a loop fabric layer located above the film backing layer and bonded thereto. The loop fabric layer has a first side and a second side opposite to the first side, and comprises a plurality of parallel warp yarns, each warp yarn being formed with a plurality of loops thereon. Each loop is respectively oriented toward the first side to define a first angle with respect to the film backing layer. The first angle ranges from about 10° to about 80°.

The second preferred embodiment of the present invention knitted loop tape, comprises: a pressure sensitive adhesive layer; a film backing layer located above the pressure sensitive adhesive layer and bonded thereto; and a loop fabric layer located above the film backing layer and bonded thereto. The loop fabric layer has a first side and a second side opposite to the first side, and comprises a plurality of parallel warp yarns, each warp yarn being formed with a plurality of loops thereon. Each of some loops along the same warp yarn is respectively oriented toward the first side to define a first angle with respect to the film backing layer. The first angle ranges from about 10° to about 80°. Each of the remaining loops along the same warp yarn is respectively oriented toward the second side to define a second angle with respect to the film backing layer. The second angle ranges from about 10° to about 80°.

The present invention also relates a hook and loop fastening system, comprising at least one loop portion made of the knitted loop tape according to the first preferred embodiment and at least one hook portion with a plurality of hooks engageable with the loops of the loop portions.

A further aspect of the present invention relates to a hook and loop fastening system, comprising at least one loop portion made of the knitted loop tape according to the second preferred embodiment and at least one hook portion with a plurality of hooks engageable with the loops of the loop portions.

Another further aspect of the present invention relates to a disposable diaper, comprising: a front waist region; a rear waist region; a crotch region extending between the front waist region and the rear waist region; a liquid-pervious topsheet; a liquid-impervious backsheet; an absorbent core inserted between the topsheet and the backsheet; the topsheet and the backsheet extending outward longitudinally beyond longitudinally opposite ends of the absorbent core and bonding together in the front waist region and the rear waist region, respectively, so as to form a pair of end flaps in each of the front waist region and the rear waist region. At least one strip of the knitted loop tape according to the first or second preferred embodiment being attached to the backsheet between one pair of the end flaps, with the warp yarns of the tape parallel to the longitudinal direction of the diaper. A tab being attached to each of the other pair of end flaps on the backsheet, each tab having a plurality of hooks which are formed thereon and face the topsheet.

The structure and objectives of the present invention can be more readily understood by persons skilled in the art from the following description of the preferred embodiments taken in conjunction with the accompanying drawings.

### BRIEF DESCRIPTION OF THE DRAWINGS

FIG. 1 is a sectional view of a conventional knitted loop tape;

FIG. 2 is a perspective view showing a segment of the conventional knitted loop tape of FIG. 1;

FIG. 3 is a sectional view of a knitted loop tape in accordance with the first preferred embodiment of the present invention;

FIG. 4 is a perspective view showing a segment of the knitted loop tape of FIG. 3;

FIG. 5 is a sectional view of a knitted loop tape in accordance with the second preferred embodiment of the present invention;

FIG. 6 is a perspective view showing a segment of the knitted loop tape of FIG. 5;

FIG. 7 is a spread view of a diaper provided with the knitted loop tape in accordance with the second preferred embodiment of the present invention; and

FIG. 8 is a perspective view of the diaper of FIG. 7.

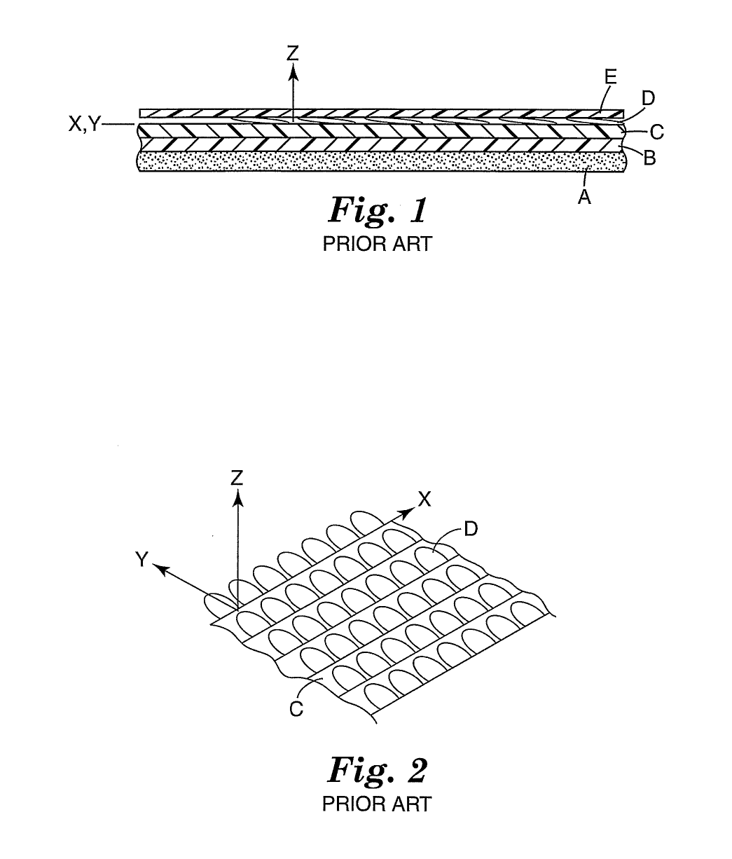

### DETAILED DESCRIPTION OF THE INVENTION

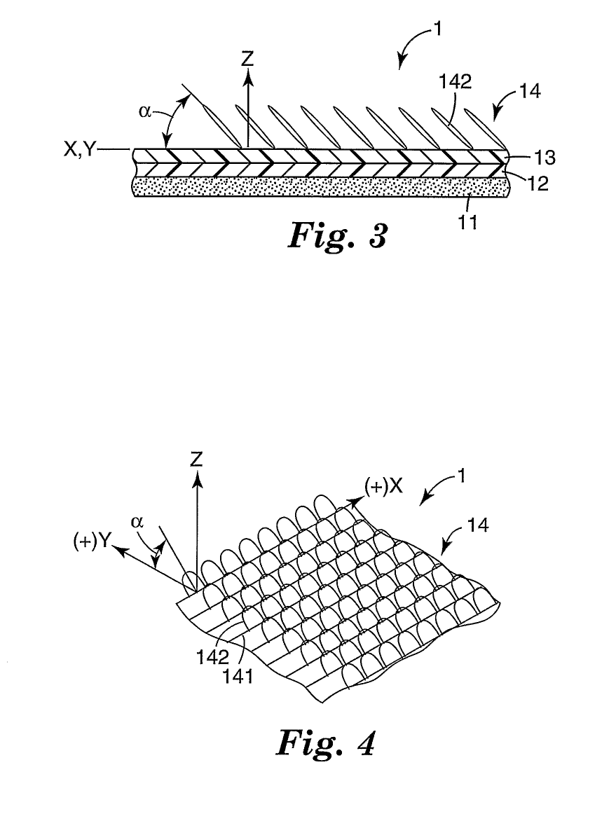

FIGS. 3 and 4 show a knitted loop tape 1 in accordance with the first preferred embodiment of the present invention. As shown, the knitted loop tape 1 comprises a pressure sensitive adhesive layer 11, a pattern printing layer 12 located above the pressure sensitive adhesive layer 11 and bonded thereto, a film backing layer 13 located above the printing layer 12 and bonded thereto, and a loop fabric layer 14 located above the film backing layer 13 and bonded thereto.

The loop fabric layer 14 lies on an X-Y plane, wherein the X axis is defined as the warp knitting direction and the Y axis is defined as the weft knitting direction. A Z axis is thus orthogonal to both the X axis and the Y-axis.

The loop fabric layer 14 comprises a plurality of parallel warp yarns 141 which are oriented in the (+)X direction. Each warp yarn 141 is formed with a plurality of loops 142 thereon.

As shown in FIGS. 3 and 4, in the current embodiment, the loops 142 are all tilted toward the (+)Y direction, or the first side of the loop fabric layer 14, with each loop 142 defining a angle α with respect to the (+)Y direction. The angle α ranges from about 10° to about 80°, preferably 20° to 60°.

In manufacturing the knitted loop tape 1, it is usually wound into a roll in the (+)X direction. When the knitted loop tape 1 is to be cut into pieces and used in a hook-and-loop fastening system, the roll of the knitted loop tape 1 has to be unwound first. Based on the angle α, when the knitted loop tape 1 is unwound (in the (−)X direction), the unwinding force is controlled such that the force does not destroy the bonding between the loop fabric layer 14 and the film backing layer 13.

It is noted that the controlled unwinding force resulted from the designated loop angle α a is similar to the effect of applying a conventional low adhesion backsize (LAB) coating. However, no LAB coating can reduce the cost in the materials, such as the release agent and solvent, and the cost in the related processes, such as the drying. Further, there will no potential contamination or voids resulted from the LAB coating.

Moreover, when the knitted loop tape 1 is used in a hook-and-loop fastening system, the designated loop angle α further increases strength of engagement between loops and hooks, which means that the peeling force for separating the hooks from the loops is increased.

It should be noted that for easily understanding the loop structure, each loop 142 is illustrated to be separate from the adjacent one, as shown in FIG. 4. However, the loops 142 along the same warp yarn 141 may overlap each other. Further, even though in the current embodiment, the angle α is defined as the angle of the loop with respect to the (+)Y direction, the loop is not necessary limited to be only tilted in the (+)Y direction but can also be tilted toward either the (+)X direction (“loop up” direction) or the (−)X direction (“loop down” direction). In the latter two cases, the angle α is defined as the angle of the loop with respect to the X-Y plane.

To further illustrate the controlled unwinding force of the knitted loop tape 1 in accordance with the first preferred embodiment of the present invention, a first test (TEST 1) was performed according to the following procedures.

TEST 1

Objective: To determine the unwinding force value of tapes in wide roll form.

Test Materials:

1. Three rolls of tape, respectively comprising a film frontal tape (FFT, Product No. KT-1985, from 3M Company), a conventional knitted loop tape (Product No. KLT, from 3M Company) and the knitted loop tape 1 (New KLT1) in accordance with the first preferred embodiment of the present invention, as listed in Table 1 and being conditioned for 24 hours in the constant temperature and humidity room; and

TABLE 1

| | FFT | KLT | New KLT1 |
|---|---|---|---|
| Basis weight (g/m²) | 22.5 | 14 + 32 + 9* | 14 + 28 + 4* |
| PSA coating weight | 19 | 27 | 32 |
| (g/m²) | | | |
| Rows/cm | x | 5.5 | 5.5 |
| Knits/cm | x | 13 | 13 |
| Yarn/count | x | L1 44/11 | L1 40/12 |
| | | L2 22/1 | L2 20/1 |
| | | L3 44/11 | L3 40/12 |

*film backing layer + loop fabric layer (with LAB layer for KLT) + pattern printing layer

2. Remove at least three laps of tape from each test roll.

Equipment:

1. Constant temperature and humidity room set at 23+/−2° C. and 50+/−5% relative humidity;

2. Instron extension tester with constant rate; and

3. Unwind apparatus equipped with six inch tongue.

Equipment Setup:

1. Instron extension tester
a. Crosshead speed: 20″/min;
b. Full scale load: Minimum 500 grams;
c. Chart speed: 10″/min;
d. Filter out;
e. Initial jaw separation: Adjust to leave approximately two inches between the upper jaw and the roll to be tested;
f. Crosshead travel: At least 6.2 inches; and
g. Zero adjust: Place the metal straight edge in the upper jaw and adjust zero.

2. Unwind apparatus
a. Clamp the unwinding apparatus into lower jaws of the constant rate of the extension tester; and
b. Make sure the spindle on the unwind apparatus turns freely.
   Procedures:

1. Testing must be done at constant temperature and humidity conditions;

2. Place a roll of tape on the spindle of the unwind apparatus, and make sure it is centered properly below the upper jaw of the extension tester;

3. Fold the tape over at free end around the metal straight edge;

4. Clamp the straight edge into the upper jaws of the constant extension tester;

5. Start the pen and chart in motion at the same time by pressing the down button; and

6. After unwinding approximately six inches of tape, shut off the chart and pen, and press return button to bring the jaws back to their initial position.

Result:

1. When the trace on the chart becomes essentially constant, count the number of small divisions (counting across the chart) on the chart from the base line to the point which represents an average reading for the mechanical unwinding of the tape from the roll; and

2. Calculate the unwinding force using the following formula:

$$Unwind ( in grams ) = \frac{\begin{matrix}Number of Small Divisions \\ from the Baseline to the Average Reading\end{matrix}}{Width of Test Roll in Inches} ⨯ \frac{Full Scale Load}{100}$$

3. The calculated unwinding force with respect to each of the film frontal tape (FFT), the conventional knitted loop tape (KLT) and the knitted loop tape 1 (New KLT1) in accordance with the first preferred embodiment of the present invention is summarized in Graph 1.

[image]

From Graph 1, it is seen that even though the KLT has a LAB layer and the New KLT1 does not have such a LAB layer, the unwinding force for the knitted loop tape 1 in accordance with the first preferred embodiment of the present invention is well controlled and smaller than that for the conventional knitted loop tape.

In addition, to show that the knitted loop tape 1 in accordance with the first preferred embodiment of the present invention has better peeling force than that of the conventional material, the test materials list in Table 1 were subjected to a second test (TEST 2) which was performed according to the following procedures.

TEST 2

Objective: To measure 135 degree peel adhesion of tape from a test surface after securing it with a 4.5 pound roller, using a 13 inch jaw separation and keeping the test panel in one position throughout the peel.

Test Materials:

1. Three rolls or pieces of tape, at least 2¼″ in the cross direction and long enough in the machine direction to cut the required number of replicates, and respectively comprising the film frontal tape (FFT, Product No. KT-1985, from 3M Company), the conventional knitted loop tape (Product No. KLT, from 3M Company) and the knitted loop tape 1 (New KLT1) in accordance with the first preferred embodiment of the present invention, as listed in Table 1 and being conditioned for 24 hours in the constant temperature and humidity room;

2. TRM-300 Double Coated Pressure Sensitive Tape (Order from Aberdeen, S. Dak. USA, 1.5″×36 yards 9579 tape #70-0000-8219-1);

3. Three 1″ wide and approximately 11″ long paper strips;

4. Three 2″×5″ pieces of test material, one being another FFT tape for adhering to the FFT tape and the other two being a hook layer (Product No. CS600, from 3M Company) for engaging with the conventional knitted loop tape (KLT) and the knitted loop tape 1 (New KLT1); and

5. 2″×5″× 1/16″ smooth steel panel or 2″×5″×⅛″ polypropylene plates (from Minnesota Plastics).

Equipment:

1. Constant temperature and humidity room set at 23+/−2° C. and 50+/−5% relative humidity;

2. Constant rate Instron extension tester with metric scale, tension load cell, clamp jaws and chart recorder;

3. 135 degree test jig with panel securer;

4. Sample cutter that holds two single-edged razor blades in parallel planes 1 inch apart;

5. 4.5 pound rubber-covered hand operated roller conforming to PSTC Test Methods Appendage B;

6. 4.5 pound rubber-covered, mechanically operated roller conforming to PSTC Test Methods Appendage B; and

7. Ruler or tape measure capable of measuring 13 inches.

Equipment Setup:

1. Instron extension tester
a. Crosshead speed: 12″/min; and
b. Initial jaw separation: Approximately 13 inches.

2. Chart recorder
a. Full scale load: 2000 grams;
b. Chart speed: 5″/minute; and
c. Filter out.

3. Test Panel
a. Make sure stainless steel test panels are clean and free of adhesive and other foreign material;
b. Clean with Diacetone alcohol and heptane as needed; and
c. If testing with polypropylene panels, use both sides of the news panels and then dispose of in an appropriate manner,
   Procedures:

1. Testing must be done at constant temperature and humidity conditions;

2. Place TRM-300 double-coated tape on one side of the steel panel; and remove the liner from the TRM-300 tape;

3. Place a 2″×5″ piece of test material, i.e., the FFT or hook layer, test side up onto the TRM-300 tape; cover with the paper strip and secure test material using a rolldown;

4. Peel the first three layers of tape from the outside of the test roll and discard;

5. Place one or more layers on the work surface; do not touch the release surface of the last layer;

6. Place one more layer down on the non-contaminated release surface of the last layer;

7. Cut 1″ wide specimens with a 1″ sample cutter; the tape will be tested in the cross direction;

8. Lift one end of the top layer of tape from a 1″ wide cut specimen, and attach a leader to approximately ¼″ of it;

9. Place the cut specimen onto the panel prepared in Steps 2 and 3; center it on the panel with the long dimension of the specimen parallel to the long dimension of the panel; do not apply any pressure to the tape;

10. Immediately roll down the tape specimen with one pass in each direction of the 4.5 pound mechanically operated roller; roll the tape lengthwise; test immediately (within 15 seconds) after rolldown is completed; only one test per panel, and rolldown only one sample at a time;

11. With the 135 degree test jig secured in the lower jaw, slide the test panel assembly into the jig slot; align the tape sample so the start of the peel occurs at the line marked on the fixture; clamp the leader into the upper jaw;

12. Start the tensile tester peeling the test specimen from the test surface; and

13. Polypropylene panels may be disposed of after testing on both sides of the panel.

Result:

1. Visually determine the average peel value obtained during the removal of the tape;

2. Count the number of small divisions from the baseline to the line representing the visually determined average;

3. Calculate the peeling force using the following formula:

$$Peel ( grams \text{/} inch ) = \frac{Number of Small Divisions ⨯ 10}{Tape Width in Inches}$$

4. The calculated 135 degree peeling force with respect to each of the film frontal tape (FFT), the conventional knitted loop tape (KLT) and the knitted loop tape 1 (New KLT1) in accordance with the first preferred embodiment of the present invention is summarized in Graph 2.

[image]

From the Graph 2, it is seen that the 135 degree peeling force for the knitted loop tape 1 in accordance with the first preferred embodiment of the present invention is larger than that for the conventional knitted loop tape.

The knitted loop tape 1 of first preferred embodiment disclosed in FIGS. 3 and 4 eliminates the need for a LAB coating. In this respect, the knitted loop tape 1 of the first preferred embodiment of the present invention may be further modified to the knitted loop tape 2 of second preferred embodiment.

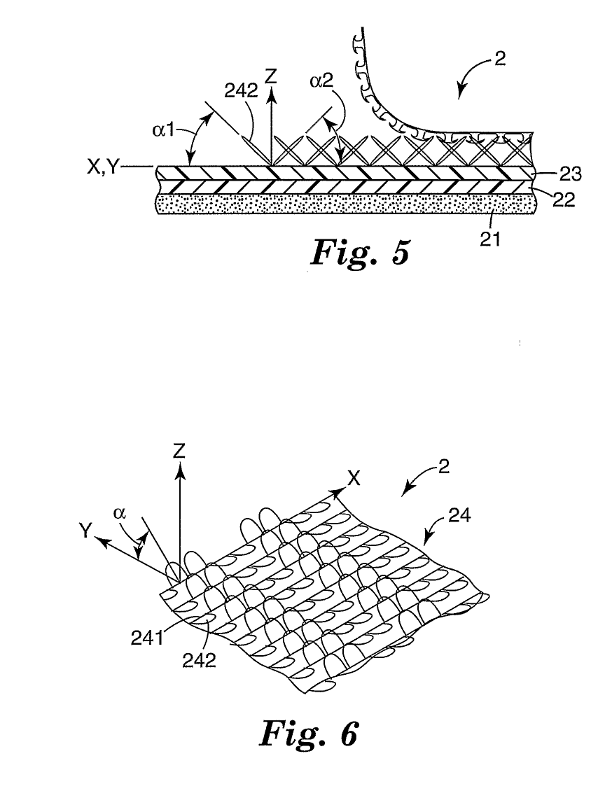

FIGS. 5 and 6 show a knitted loop tape 2 in accordance with the second preferred embodiment of the present invention. As shown, the knitted loop tape 2 comprises a pressure sensitive adhesive layer 21, a pattern printing layer 22 located above the pressure sensitive adhesive layer 21 and bonded thereto, a film backing layer 23 located above the printing layer 22 and bonded thereto, and a loop fabric layer 24 located above the film backing layer 23 and bonded thereto.

The loop fabric layer 24 lies on an X-Y plane, wherein the X axis is defined as the warp knitting direction and the Y axis is defined as the weft knitting direction. A Z axis is thus orthogonal to both the X axis and the Y-axis.

The loop fabric layer 24 comprises a plurality of parallel warp yarns 241 which are oriented in the (+)X direction. Each warp yarn 241 is formed with a plurality of loops 242 thereon.

As shown in FIGS. 5 and 6, in the current embodiment, some of the loops 242 along the same warp yarn 241 are tilted in pairs toward the (+)Y direction, or the first side of the loop fabric layer 24, with each loop 242 defining a first angle α1 with respect to the (+)Y direction, and the remaining loops 242 along the same warp yarn 241 are tilted in pairs toward the (−)Y direction, or the second side of the loop fabric layer 24, with each loop defining a second angle α2 with respect to the (−)Y direction. The angles α1 and α2 range from about 10° to about 80°, preferably 20° to 60°. As shown in FIGS. 5 and 6, the adjacent loop pairs are tilted in opposite directions.

Similar to the first preferred embodiment, in manufacturing the knitted loop tape 2, it is usually wound into a roll in the (+)X direction. When the knitted loop tape 2 is to be cut into pieces and used in a hook-and-loop fastening system, the roll of the knitted loop tape 2 has to be unwound first. Based on the angles α1 and α2, when the knitted loop tape 2 is unwound (in the (−)X direction), the unwinding force is controlled such that the force does not destroy the bonding between the loop fabric layer 24 and the film backing layer 23. The peeling force of the knitted loop tape 2 is larger than that of the conventional knitted loop tape.

It should be noted that for easily understanding the loop structure, each loop 242 is illustrated to be separate from the adjacent one, as shown in FIG. 6. However, the loops 242 of the same pair along the same warp yarn 241 may overlap each other. Further, even though in the current embodiment, the first angle α1 is defined as the angle of the loop with respect to the (+)Y direction, and the second angle α2 is defined as the angle of the loop with respect to the (−)Y direction, the loop is not necessary limited to be only tilted in the (+)Y direction or the (−)Y direction, but can also be tilted toward either the (+)X direction (“loop up” direction) or the (−)X direction (“loop down” direction). In the latter two cases, each of the angles α1 and α2 is defined as the angle of the loop with respect to the X-Y plane.

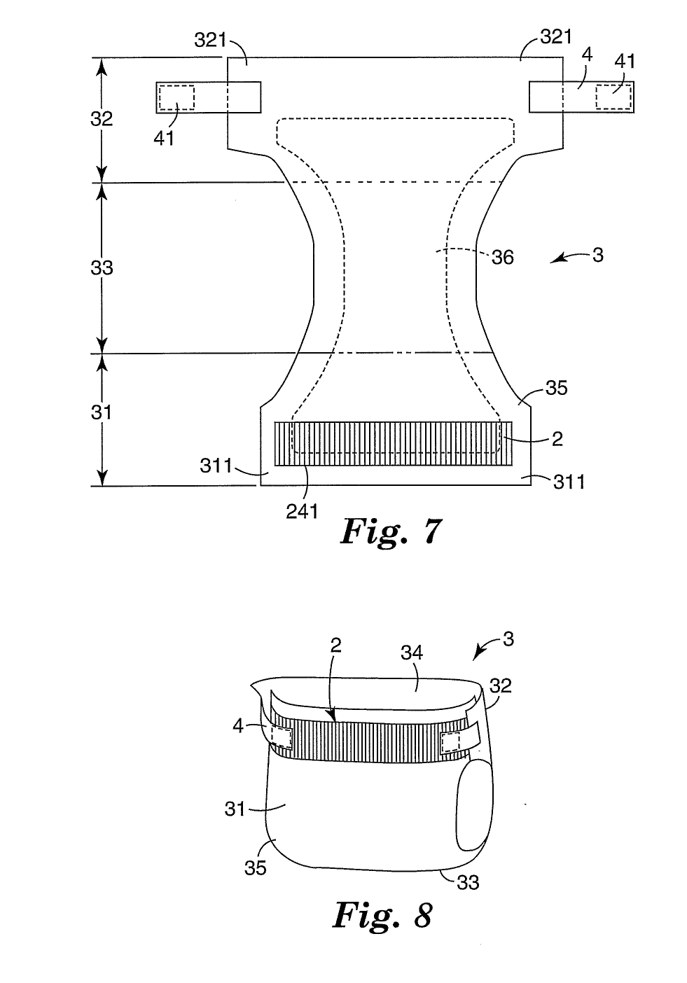

FIGS. 7 and 8 show a diaper 3 provided with a knitted loop tape 2 in accordance with the second preferred embodiment of the present invention. As shown, the diaper 3 comprises a front waist region 31, a rear waist region 32 and a crotch region 33 extending between the front waist region 31 and the rear waist region 32, as well as a liquid-pervious topsheet 34, a liquid-impervious backsheet 35, and an absorbent core 36 inserted between the topsheet 34 and the backsheet 35.

As shown in FIG. 7, the topsheet 34 and the backsheet 35 extend outward longitudinally beyond the longitudinally opposite ends of the absorbent core 36 and bond together in the front waist region 31 and the rear waist region 32, respectively. A first pair of end flaps 311 is thus formed in the front waist region 31 and a second pair of end flaps 321 is formed in the rear waist region 32.

To install the hook-and-loop fastening system to the diaper 3, a strip of the knitted loop tape 2 in accordance with the second preferred embodiment of the present invention is attached to the backsheet 35 between the end flaps 311, with the warp yarns 241 of the tape 2 parallel to the longitudinal direction of the diaper 3. Further, a tab 4, having a hook portion 41 with a plurality of hooks formed thereon (e.g., the hooks shown in FIG. 5) and facing the topsheet 34, is attached to each end flap 321, on the backsheet 35. Accordingly, by engaging the hooks 41 of the tab 4 on each end flap 321 with the knitted loop tape 2, the diaper 3 can be folded on a user's body, as shown in FIG. 8.

According to the diaper 3 of the present invention, since the loops 242 of the knitted loop tape 2 are substantially evenly tilted toward both directions, the force for peeling each of the opposite tabs 4 from the tape 2 is similar to each other.

To further prove the balanced peeling force on each side of the diaper 3 with the use of the knitted loop tape 2 in accordance with the second preferred embodiment of the present invention, a third test (TEST 3) is introduced and performed according to the following procedures.

TEST 3

Objective: To measure the shear strength of hook and loop touch fasteners using a recording constant rate of extension tensile testing machine.

Test Materials:

1. A 1″ MD (machine direction)×3″ CD (cross direction) conventional knitted loop tape (Product No. KLT, from 3M Company) and a 1″ MD×3″ CD knitted loop tape 2 (New KLT2) in accordance with the second preferred embodiment of the present invention, as listed in Table 2 and being conditioned for 24 hours in the constant temperature and humidity room; and

2. A 1″ MD×½″ CD hook material hook layer (Product No. CS600, from 3M Company).

TABLE 2

| | KLT | New KLT2 |
|---|---|---|
| Fabric basis weight (g/m²) | 32 | 30 |
| Rows/cm | 5.5 | 5.5 |
| Knits/cm | 13 | 13 |
| Yarn/count | L1 44/11 | L1 40/12 |
| | L2 22/1 | L2 20/1 |
| | L3 44/11 | L3 40/12 |
| Adhesive coating weight (g/m²) | 8 | 8 |
| Printing layer thickness (μm) | 15 | 15 |

Equipment:

1. Instron extension tester with constant rate;

2. 1″ razor blade cutter and single razor blade; and

3. A roller that is 11¼ pounds.

Equipment Setup:

1. Full scale load should be 20,000 grams, and depending on aggressive test material, up to 50,000 grams; adjust only if result is not between 20% and 80% of full scale load;

2. Chart speed 20″/minute;

3. Crosshead speed is 12″/minute;

4. Initial jaw separation is 3″.

Procedures:

1. Cut out samples of both Hook 1″ MD×½″ CD; use strapping tape to reinforce the back of each sample, and create a leader;

2. Carefully align the Hook material on the center of Loop material;

3. Using the 11¼ pounds roller, push the roller without adding weight over the entire length of the joined strips; then pull it back over entire length; this constitutes one cycle; a continuous uninterrupted back and forth motion of the roller, without lifting it from the sample; each cycle should take about two seconds; and perform five continuous cycles on each sample;

4. Position the clamps of the tensile tester so they are 3″ apart; place the free ends of the sample in the clamps; the hook sample should be in the upper clamp; the sample should be centered and straight up and down in the clamps; and

5. Observe the chart or computer screen and end the test when it reaches its maximum peak.

Result:

1. Report the maximum peak in grams; and

2. The observed 90 degree peeling force on the left side and right side of the diaper with respect to each of the conventional knitted loop tape (KLT) and the knitted loop tape 2 (New KLT2) in accordance with the second preferred embodiment of the present invention is summarized in Graph 3.

[image]

From the disclosure of Graph 3, it is seen that with the use of the knitted loop tape 2 of the present invention, the peeling forces on both sides of the diaper 3 are well balanced.

The above descriptions have clearly illustrated the important features, operational methods and applications of the present invention. Although the invention has been described with reference to the preferred embodiments, it will be obvious to persons skilled in the art that various changes and modifications may be made without departing from the scope of the invention as recited in the claims.

1. A knitted loop tape, comprising:
a pressure sensitive adhesive layer;
a film backing layer located above said pressure sensitive adhesive layer and bonded thereto; and
a loop fabric layer located above said film backing layer and bonded thereto, said loop fabric layer having a first side and a second side opposite to said first side, and comprising a plurality of parallel warp yarns, each warp yarn being formed with a plurality of loops thereon;
wherein each loop is respectively oriented toward said first side to define a first angle with respect to said film backing layer and said first angle ranges from about 10° to about 80°.

2. The knitted loop tape according to Claim 1, further comprising a printing layer between said film backing layer and said pressure sensitive adhesive layer and bonded thereto.

3. The knitted loop tape according to claim 1, wherein said first angle is preferably from about 20° to about 60°.

4. The knitted loop tape according to Claim 1, wherein said first angle for each loop is approximately the same.

5. A knitted loop tape, comprising:
a pressure sensitive adhesive layer;
a film backing layer located above said pressure sensitive adhesive layer and bonded thereto; and
a loop fabric layer located above said film backing layer and bonded thereto, said loop fabric layer having a first side and a second side opposite to said first side, and comprising a plurality of parallel warp yarns, each warp yarn being formed with a plurality of loops thereon;
wherein each of some loops along the same warp yarn is respectively oriented toward said first side to define a first angle with respect to said film backing layer and said first angle ranges from about 10° to about 80°, and each of the remaining loops along the same warp yarn is respectively oriented toward said second side to define a second angle with respect to said film backing layer and said second angle ranges from about 10° to about 80°.

6. The knitted loop tape according to Claim 5, further comprising a printing layer between said film backing layer and said pressure sensitive adhesive layer and bonded thereto.

7. The knitted loop tape according to Claim 5, wherein said first angle and said second angle are preferably from about 20° to about 60°.

8. The knitted loop tape according to Claim 5, wherein said first angle for said each of some loops along the same warp yarn is approximately the same, and said second angle for said each of the remaining loops along the same warp yarn is approximately the same.

9. A hook and loop fastening system comprising at least one loop portion made of the knitted loop tape according to claim 1 and at least one hook portion with a plurality of hooks engageable with said loops of said loop portions.

10. A hook and loop fastening system comprising at least one loop portion made of the knitted loop tape according to claim 5 and at least one hook portion with a plurality of hooks engageable with said loops of said loop portions.

11. (canceled)

12. A disposable garment, comprising:
a front waist region;
a rear waist region;
a crotch region extending between said front waist region and said rear waist region;
a liquid-pervious topsheet;
a liquid-impervious backsheet;
an absorbent core inserted between said topsheet and said backsheet;
said topsheet and said backsheet extending outward longitudinally beyond longitudinally opposite ends of said absorbent core and bonding together in said front waist region and said rear waist region, respectively, so as to form a pair of end flaps in each of said front waist region and said rear waist region;
at least one strip of a knitted loop tape being attached to said backsheet between one pair of said end flaps, with said warp yarns of said tape parallel to the longitudinal direction of said diaper, said knitted loop tape comprising
a pressure sensitive adhesive layer;
a film backing layer located above said pressure sensitive adhesive layer and bonded thereto; and
a loop fabric layer located above said film backing layer and bonded thereto, said loop fabric layer having a first side and a second side opposite to said first side, and comprising a plurality of parallel warp yarns, each warp yarn being formed with a plurality of loops thereon:
wherein each loop is respectively oriented toward said first side to define a first angle with respect to said film backing layer and said first angle ranges from about 10° to about 80°; and a tab being attached to each of the other pair of end flaps on the backsheet, each tab having a plurality of hooks which are formed thereon and face the topsheet.

13. A disposable garment, comprising:
a front waist region;
a rear waist region;
a crotch region extending between said front waist region and said rear waist region;
a liquid-pervious topsheet;
a liquid-impervious backsheet;
an absorbent core inserted between said topsheet and said backsheet;
said topsheet and said backsheet extending outward longitudinally beyond longitudinally opposite ends of said absorbent core and bonding together in said front waist region and said rear waist region, respectively, so as to form a pair of end flaps in each of said front waist region and said rear waist region:
at least one strip of a knitted loop tape being attached to said backsheet between one pair of said end flaps, with said warp yarns of said tape parallel to the longitudinal direction of said diaper, said knitted loop tape comprising
a pressure sensitive adhesive layer;
a film backing layer located above said pressure sensitive adhesive layer and bonded thereto; and
a loop fabric layer located above said film backing layer and bonded thereto, said loop fabric layer having a first side and a second side opposite to said first side, and comprising a plurality of parallel warp yarns, each warp yarn being formed with a plurality of loops thereon;
wherein each of some loops along the same warp yarn is respectively oriented toward said first side to define a first angle with respect to said film backing layer and said first angle ranges from about 10° to about 80°, and each of the remaining loops along the same warp yarn is respectively oriented toward said second side to define a second angle with respect to said film backing layer and said second angle ranges from about 10° to about 80°; and
a tab being attached to each of the other pair of end flaps on the backsheet, each tab having a plurality of hooks which are formed thereon and face the topsheet.
